(19) United States
(12) Patent Application Publication
(10) Pub. No.: US 2026/0069832 A1
(43) Pub. Date: Mar. 12, 2026

(54) Medical-Device Navigation Systems and Methods with Electromagnetoresponsive Elements

(71) Applicant: Bard Access Systems, Inc., Salt Lake City, UT (US)

(72) Inventor: Shayne Messerly, Kaysville, UT (US)

(21) Appl. No.: 18/829,126

(22) Filed: Sep. 9, 2024

(51) Int. Cl. A61M 25/01

(52) U.S. Cl. CPC A61M 25/0127; A61M 25/0108; A61M 2025/0166; A61M 2205/0272; A61M 2205/502; A61M 2210/12

(57) ABSTRACT

A medical-device navigation system includes, in some embodiments, an electromagnetoresponsive (“EMR”) element, a magnetic interrogator, and a console. The magnetic interrogator generates an external magnetic field, transduces resonance-based responses of the EMR element thereto, and provides response data for the EMR element as an elongate portion of a medical device including the EMR element moves through the external magnetic field. The console instantiates medical-device navigation processes including a data-acquisition process, a triangulation process, and a plotting process for navigating the elongate portion of the medical device as it is advanced through the external magnetic field to a target location. The data-acquisition process acquires the response data from the magnetic interrogator over time, the triangulation process triangulates the EMR element with respect to magnetic sensors of the magnetic interrogator over time, and the plotting process plots a location of the EMR element on a display screen of the console over time.

### BACKGROUND

Navigation and appropriate placement of medical devices including needles, catheters, and the like are important for patient (and clinician) safety and clinical outcomes. Ultrasound methods are typically used for navigation of needles into patient vasculatures and determination of whether distal tips of the needles are appropriately placed in their target vascular locations. However, such ultrasound methods are highly dependent upon vascular visibility in ultrasound images, needle visibility in the ultrasound images, and operator experience. Fluoroscopic methods are typically used for navigation of catheters through patient vasculatures and determination of whether distal tips of the catheters are appropriately placed in their target vascular locations. However, such fluoroscopic methods expose patients and, possibly, their clinicians to harmful X-ray radiation. Moreover, the patients can be exposed to potentially harmful contrast media needed for the fluoroscopic methods. As such, alternative navigation means for navigating medical devices are needed.

Disclosed herein are medical-device navigation systems and methods that utilize electromagnetoresponsive (“EMR”) elements to address at least the foregoing.

### SUMMARY

Disclosed herein is a medical-device navigation system including, in some embodiments, an EMR element, a magnetic interrogator, and a console. The EMR element is in an elongate portion of a medical device. The EMR element is responsive to an external magnetic field. The magnetic interrogator is configured to generate the external magnetic field, transduce resonance-based responses of the EMR element to the external magnetic field with a plurality of magnetic sensors, and provide response data for the EMR element as the elongate portion of the medical device including the EMR element moves through the external magnetic field. The console has electronic components and circuitry including memory and one or more processors. The memory includes executable instructions configured to instantiate medical-device navigation processes upon execution by the processor(s) for navigating the elongate portion of the medical device including the EMR element as it is advanced to a target location. The medical-device navigation processes include a data-acquisition process that utilizes data-acquisition logic for acquiring the response data from the magnetic interrogator over time and writing the response data to the memory of the console. The medical-device navigation processes also include a triangulation process that utilizes triangulation logic with the response data for triangulating the EMR element with respect to each of the magnetic sensors over time and writing three-dimensional (“3D”) location data to the memory of the console. The 3D location data is set in a coordinate system established by the magnetic sensors. The medical-device navigation processes also include a plotting process that utilizes plotting logic with the 3D location data for plotting a location of the EMR element on a display screen of the console over time as the elongate portion of the medical device is advanced through the external magnetic field to the target location.

In some embodiments, the target location is in a body of a patient. The plotting process further plots the location of the EMR element over a patient avatar on the display screen of the console in real-time as the elongate portion of the medical device including the EMR element is advanced through the body of the patient to the target location.

In some embodiments, the medical device is a central venous catheter (“CVC”), and the target location is a superior vena cava (“SVC”). The plotting process further plots the location of the EMR element over the patient avatar on the display screen of the console over time as the elongate portion of the medical device including the EMR element is advanced through a vasculature of the patient including a right internal jugular vein, a right brachiocephalic vein, and into the SVC.

In some embodiments, the medical device is a stylet configured to be disposed in a primary lumen of a CVC such that their distal ends are approximately coterminal. The target location is an SVC. The plotting process further plots the location of the EMR element over the patient avatar on the display screen of the console over time as the elongate portion of the medical device including the EMR element is advanced through a vasculature of the patient including a right internal jugular vein, a right brachiocephalic vein, and into the SVC.

In some embodiments, the medical device is a peripherally inserted central catheter (“PICC”), and the target location is an SVC. The plotting process further plots the location of the EMR element over the patient avatar on the display screen of the console over time as the elongate portion of the medical device including the EMR element is advanced through a vasculature of the patient including a right basilic vein, a right axillary vein, a right subclavian vein, a right brachiocephalic vein, and into the SVC.

In some embodiments, the medical device is a stylet configured to be disposed in a primary lumen of a PICC such that their distal ends are approximately coterminal. The target location is an SVC. The plotting process further plots the location of the EMR element over the patient avatar on the display screen of the console over time as the elongate portion of the medical device including the EMR element is advanced through a vasculature of the patient including a right basilic vein, a right axillary vein, a right subclavian vein, a right brachiocephalic vein, and into the SVC.

Also disclosed herein is a medical-device navigation system including, in some embodiments, a pair of EMR elements, a magnetic interrogator, and a console. The pair of EMR elements is distributed between a pair of medical devices such that a first EMR element is in an elongate portion of a first medical device and a second EMR element is in any portion of a second medical device. Each EMR element of the EMR elements is responsive to an external magnetic field. The magnetic interrogator is configured to generate the external magnetic field, transduce resonance-based responses of the EMR elements to the external magnetic field with a plurality of magnetic sensors, and provide response data for the EMR elements as the elongate portion of the first medical device including the first EMR element moves through the external magnetic field. The console has electronic components and circuitry including memory and one or more processors. The memory includes executable instructions configured to instantiate medical-device navigation processes upon execution by the processor(s) for navigating the elongate portion of the first medical device including the first EMR element as it is advanced to a target location.

The medical-device navigation processes include a data-acquisition process that utilizes data-acquisition logic for acquiring the response data from the magnetic interrogator over time and writing the response data to the memory of the console. The medical-device navigation processes also include a triangulation process that utilizes triangulation logic with the response data for triangulating the EMR elements with respect to each of the magnetic sensors over time and writing 3D location data to the memory of the console. The 3D location data is set in a coordinate system established by the magnetic sensors. The medical-device navigation processes also include a vectoring process that utilizes vectoring logic with the 3D location data for determining absolute location and velocity vectors for the EMR elements in the coordinate system established by the magnetic sensors as well as relative location and velocity vectors between the first EMR element and the second EMR element therefrom and writing such vectors to the memory of the console. The medical-device navigation processes also include a vector-analysis process that utilizes vector-analysis logic with some combination of the vectors for determining any movement of the first EMR element in the elongate portion of the first medical device through the external magnetic field with respect to the target location.

In some embodiments, the first medical device is a CVC or PICC with the first EMR element about a distal tip of a catheter tube of the CVC or PICC. The second medical device is a securement device for securing an extracorporeal portion of the CVC or PICC to a surface of a patient. The second EMR element is incorporated into a catheter-hub holder of the securement device. The target location is an SVC in a body of the patient.

In some embodiments, determining any movement of the first EMR through the external magnetic field with respect to the target location includes determining any displacement of the distal tip of the CVC or PICC from the SVC by way of the relative location or velocity vectors between the first EMR element in the distal tip of the CVC or PICC and the second EMR element in the catheter-hub holder of the securement device.

In some embodiments, the medical-device navigation processes further include displaying any displacement of the distal tip of the CVC or PICC from the SVC with a graphical representation of the elongate portion of the CVC or PICC over a patient avatar on a display screen of the console.

In some embodiments, the first medical device is a CVC or PICC with the first EMR element about a distal tip of a catheter tube of the CVC or PICC. The second medical device is a catheter introducer for introducing the elongate portion of the CVC or PICC to a vasculature of a patient. The second EMR element is incorporated into a distal tip of the catheter introducer. The target location is an SVC in a body of the patient.

In some embodiments, determining any movement of the first EMR element through the external magnetic field with respect to the target location includes determining advancement of the distal tip of the CVC or PICC toward the SVC by way of the relative location or velocity vectors between the first EMR element in the distal tip of the CVC or PICC and the second EMR element in the distal tip of the catheter introducer.

In some embodiments, the relative location or velocity vectors between the first EMR element in the distal tip of the CVC or PICC and the second EMR element in the distal tip of the catheter introducer need not be with respect to a common time. The vector-analysis process utilizes the absolute location or velocity vectors for the second EMR element in the distal tip of the catheter introducer at a time prior to removing the introducer catheter from the patient. The vector-analysis process utilizes the absolute location or velocity vectors for the first EMR element in the distal tip of the CVC or PICC while advancing the distal tip of the CVC or PICC toward the SVC at a time after removing the introducer catheter from the patient.

In some embodiments, the medical-device navigation processes further include displaying advancement of the distal tip of the CVC or PICC toward the SVC with a graphical representation of the elongate portion of the CVC or PICC over a patient avatar on a display screen of the console.

In some embodiments, the first medical device is a needle with the first EMR element about a distal tip of a needle shaft of the needle. The second medical device is an ultrasound probe for ultrasound imaging a vasculature of a patient. The second EMR element is incorporated into a probe head of the ultrasound probe. The target location is a blood vessel in the vasculature of the patient.

In some embodiments, determining any movement of the first EMR through the external magnetic field with respect to the target location includes determining advancement of the distal tip of the needle toward the blood vessel by way of the relative location or velocity vectors between the first EMR element in the distal tip of the needle, the second EMR element in the probe head of the ultrasound probe, and a triangulated location of the blood vessel below the probe head of the ultrasound probe determined by the ultrasound imaging.

In some embodiments, the medical-device navigation processes further includes displaying an ultrasound image on a display screen of the console while advancing the distal tip of the needle toward the blood vessel below the probe head of the ultrasound probe. A blood-vessel segment of the ultrasound image corresponding to the blood vessel below the probe head of the ultrasound probe is visually indicated in the ultrasound image when the needle is aligned therewith.

In some embodiments, the first medical device is a Huber needle with the first EMR element about a distal tip of a needle shaft of the Huber needle. The second medical device is a venous access port. The second EMR element, optionally, with one or more additional EMR elements, is incorporated into the venous access port around a septum thereof. The target location is a reservoir of the venous access port covered by the septum.

In some embodiments, determining any movement of the first EMR through the external magnetic field with respect to the target location includes advancement of the distal tip of the Huber needle toward the septum of the venous access port by way of the relative location or velocity vectors between the first EMR element in the distal tip of the Huber needle, the second EMR element in the venous access port around the septum, and, optionally, the one or more additional EMR elements in the venous access port around the septum.

In some embodiments, the medical-device navigation processes further include displaying advancement of the distal tip of the Huber needle toward the septum of the venous access port with graphical representations of the distal tip of the Huber needle and the venous access port on a display screen of the console.

These and other features of the concepts provided herein will become more apparent to those of skill in the art in view of the accompanying drawings and following description, which describe particular embodiments of such concepts in greater detail.

### BRIEF DESCRIPTION OF DRAWINGS

FIG. 1 is a block diagram of a first medical-device navigation system with one or more EMR elements in accordance with some embodiments.

FIG. 2 is a block diagram of a second medical-device navigation system with the EMR element(s) in accordance with some embodiments.

FIG. 3 illustrates the second medical-device navigation system with at least one of a stylet or a catheter as a medical device including the EMR element(s) in accordance with some embodiments.

FIG. 4 illustrates a simplified view of a distal end portion of an elongate portion of the medical device including the EMR element(s) in accordance with some embodiments.

FIG. 5A illustrates the catheter including the EMR element(s) in accordance with some embodiments.

FIG. 5B illustrates the stylet including the EMR element(s) in accordance with some embodiments.

FIG. 5C illustrates a catheter introducer including the EMR element(s) in accordance with some embodiments.

FIG. 5D illustrates a securement device including the EMR element(s) in accordance with some embodiments.

FIG. 5E illustrates a needle including the EMR element(s) in accordance with some embodiments.

FIG. 5F illustrates an ultrasound probe including the EMR element(s) in accordance with some embodiments.

FIG. 5G illustrates a Huber needle including the EMR element(s) in accordance with some embodiments.

FIG. 5H illustrates a venous access port including the EMR element(s) in accordance with some embodiments.

FIG. 6A illustrates the second medical-device navigation system with the stylet and catheter in use on a patient during a medical procedure, at least one of the stylet or the catheter being the medical device including the EMR element(s) in accordance with some embodiments.

FIG. 6B further illustrates the second medical-device navigation system with the stylet and catheter in use on the patient during the medical procedure, the stylet and catheter being advanced further into the patient in accordance with some embodiments.

FIG. 7A illustrates the second medical-device navigation system with the catheter and securement device in use on a patient during a medical procedure, both of the catheter and the securement device being medical devices including the EMR element(s) in accordance with some embodiments.

FIG. 7B further illustrates the second medical-device navigation system with the catheter and securement device in use on the patient during the medical procedure, the catheter being withdrawn or displaced from its target location in the patient in accordance with some embodiments.

FIG. 8A illustrates the second medical-device navigation system with the catheter introducer and catheter in use on a patient during a medical procedure, both of the catheter introducer and the catheter being medical devices including the EMR element(s) in accordance with some embodiments.

FIG. 8B further illustrates the second medical-device navigation system with the catheter introducer removed and the catheter in use on the patient during the medical procedure, the catheter being advanced further into the patient in accordance with some embodiments.

FIG. 9A illustrates the second medical-device navigation system with the needle and ultrasound probe in use on a patient during a medical procedure, both of the needle and the ultrasound probe being medical devices including the EMR element(s) in accordance with some embodiments.

FIG. 9B further illustrates the second medical-device navigation system with the needle and ultrasound probe in use on the patient during a medical procedure, the needle accessing a blood vessel of the patient in accordance with some embodiments.

FIG. 10A illustrates the second medical-device navigation system with a Huber needle in use on a patient with a venous access port, both of the Huber needle and the venous access port being medical devices including the EMR element(s) in accordance with some embodiments.

FIG. 10B illustrates the second medical-device navigation system with the Huber needle in use on the patient with the venous access port, the Huber needle aligned with a septum of the venous access port in accordance with some embodiments.

FIG. 10C illustrates the second medical-device navigation system with the Huber needle in use on the patient with the venous access port, the Huber needle accessing a reservoir covered by the septum of the venous access port in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

“Proximal” is used to indicate a portion, section, piece, element, or the like of a medical device intended to be near or relatively nearer to a clinician when the medical device is used on a patient. For example, a “proximal portion” or “proximal section” of the medical device includes a portion or section of the medical device intended to be near the clinician when the medical device is used on the patient. Likewise, a “proximal length” of the medical device includes a length of the medical device intended to be near the clinician when the medical device is used on the patient. A “proximal end” of the medical device is an end of the medical device intended to be near the clinician when the medical device is used on the patient. The proximal portion, the proximal section, or the proximal length of the medical device need not include the proximal end of the medical device. Indeed, the proximal portion, the proximal section, or the proximal length of the medical device can be short of the proximal end of the medical device. However, the proximal portion, the proximal section, or the proximal length of the medical device can include the proximal end of the medical device. Should context not suggest the proximal portion, the proximal section, or the proximal length of the medical device includes the proximal end of the medical device, or if it is deemed expedient in the following description, “proximal portion,” “proximal section,” or “proximal length” can be modified to indicate such a portion, section, or length includes an end portion, an end section, or an end length of the medical device for a “proximal end portion,” a “proximal end section,” or a “proximal end length”of the medical device, respectively.

“Distal” is used to indicate a portion, section, piece, element, or the like of a medical device intended to be near, relatively nearer, or even in a patient when the medical device is used on the patient. For example, a “distal portion” or “distal section” of the medical device includes a portion or section of the medical device intended to be near, relatively nearer, or even in the patient when the medical device is used on the patient. Likewise, a “distal length” of the medical device includes a length of the medical device intended to be near, relatively nearer, or even in the patient when the medical device is used on the patient. A “distal end” of the medical device is an end of the medical device intended to be near, relatively nearer, or even in the patient when the medical device is used on the patient. The distal portion, the distal section, or the distal length of the medical device need not include the distal end of the medical device. Indeed, the distal portion, the distal section, or the distal length of the medical device can be short of the distal end of the medical device. However, the distal portion, the distal section, or the distal length of the medical device can include the distal end of the medical device. Should context not suggest the distal portion, the distal section, or the distal length of the medical device includes the distal end of the medical device, or if it is deemed expedient in the following description, “distal portion,” “distal section,” or “distal length” can be modified to indicate such a portion, section, or length includes an end portion, an end section, or an end length of the medical device for a “distal end portion,” a “distal end section,” or a “distal end length”of the medical device, respectively.

“Navigation” of a medical device or an elongate portion thereof by way of any medical-device navigation system or method disclosed herein encompasses tracking of the medical device, guidance of the medical device, or both tracking and guidance of the medical device with the medical-device navigation system or method, as the case might be. In an example, navigation of the medical device or an elongate portion thereof can include on-screen tracking of the medical device with the medical-device navigation system and on-screen guidance of the medical device with the medical-device navigation system by way of one or more visual indicators on the display screen. In another example, navigation of the medical device or an elongate portion thereof can include on-screen tracking of the medical device with the medical-device navigation system and clinician-based guidance of the medical device based upon the on-screen tracking of the medical device.

“Location” is used to indicate, for example, a location of a medical device or an elongate portion thereof in the medical device in some spatial or coordinate reference system such as that established by the magnetic sensors of the magnetic interrogator disclosed herein.

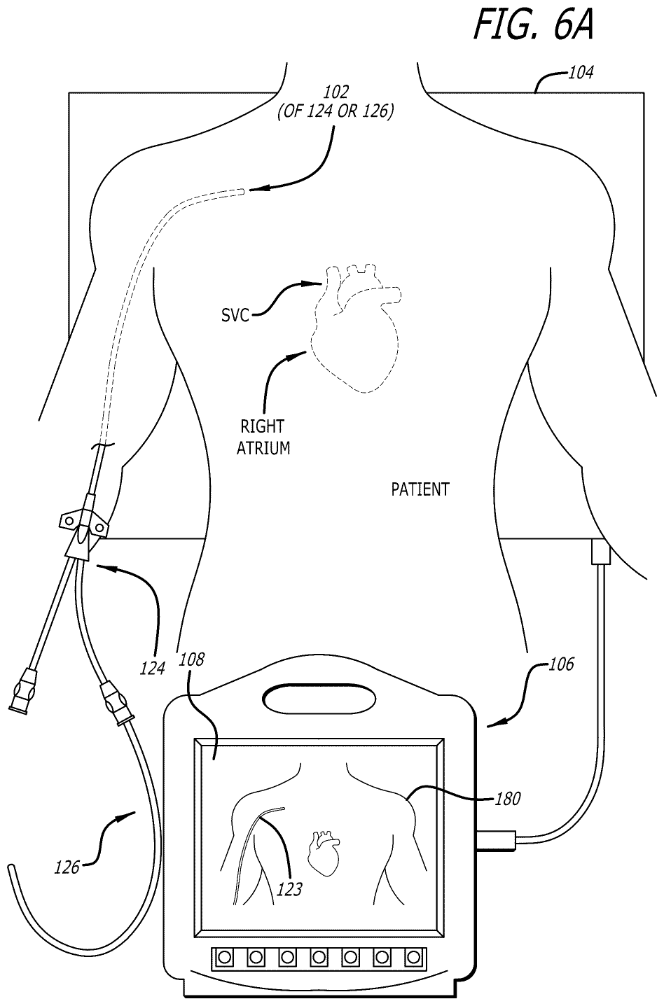

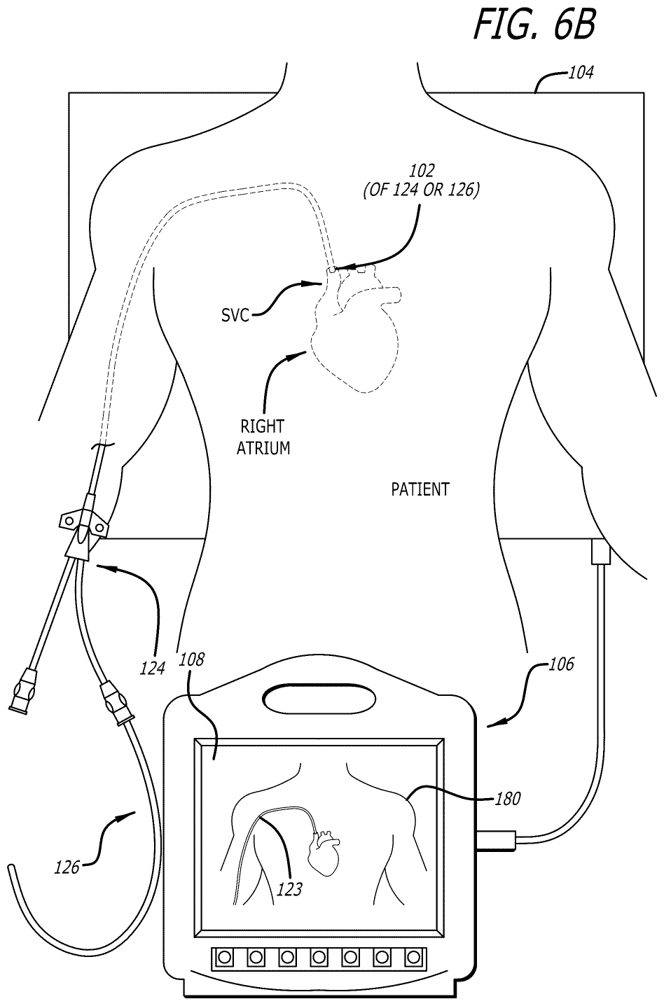

“Orientation” is used to indicate an orientation of a medical device or an elongate portion thereof in its location. By way of example, a distal tip of the elongate medical device graphically represented in FIG. 6B is in the SVC with an orientation toward the right atrium of the heart.

“Logic” can be hardware, firmware, or software configured to perform one or more functions. As hardware, logic can include circuitry having data processing functionality, data storage functionality, or both. An example of such circuitry can include, but is not limited to, a hardware processor (e.g., a microprocessor, one or more processor cores, a digital-signal processor [“DSP”], a programmable gate array [“PGA”], a microcontroller, an application-specific integrated circuit [“ASIC”], etc.) or semiconductor memory. As firmware, the logic can be stored in persistent storage. As software, logic can include one or more processes, instances, Application Programming Interfaces (“APIs”), subroutines, functions, applets, servlets, or routines. Logic can also include source code, object code, a shared library, a dynamic link library (“DLL”), or even one or more instructions. Such software can be stored in any type of suitable non-transitory computer-readable storage medium or transitory storage medium (e.g., electrical, optical, acoustical, or any other form of propagated signal including carrier waves, infrared signals, or digital signals). An example of a non-transitory computer-readable storage medium can include, but is not limited to, a programmable circuit; non-persistent storage such as volatile memory (e.g., any type of random-access memory [“RAM”]); or persistent storage such as non-volatile memory (e.g., read-only memory [“ROM”], power-backed RAM, flash memory, phase-change memory, etc.), a solid-state drive, a hard-disk drive, an optical-disc drive, or a portable memory device.

Unless defined otherwise, all technical and scientific terms used herein have the same meaning as commonly understood by those of ordinary skill in the art.

As set forth above, alternative navigation means for navigating and placing medical devices are needed. Accordingly, medical-device navigation systems and methods with EMR elements are disclosed herein as alternative navigation means for navigating or placing medical devices. Medical-device navigation systems are initially described followed by some medical devices including EMR elements, the medical device navigation systems thereby including the medical device in at least some embodiments. Subsequently, various modalities of the medical-device navigation systems for navigating or placing those medical devices are described. Broadly, such modalities can be divided among the following: 1) One or more EMR elements, for example, a single EMR element, of a medical device is used for navigating or placing that medical device as it is moved through an external magnetic field to a target location. 2) Two or more EMR elements, for example, a pair of EMR elements, distributed between two or more medical devices, respectively, are used together for navigating or placing one of the two or more medical devices as that medical device is moved through the external magnetic field to a target location.

### Medical-Device Navigation Systems

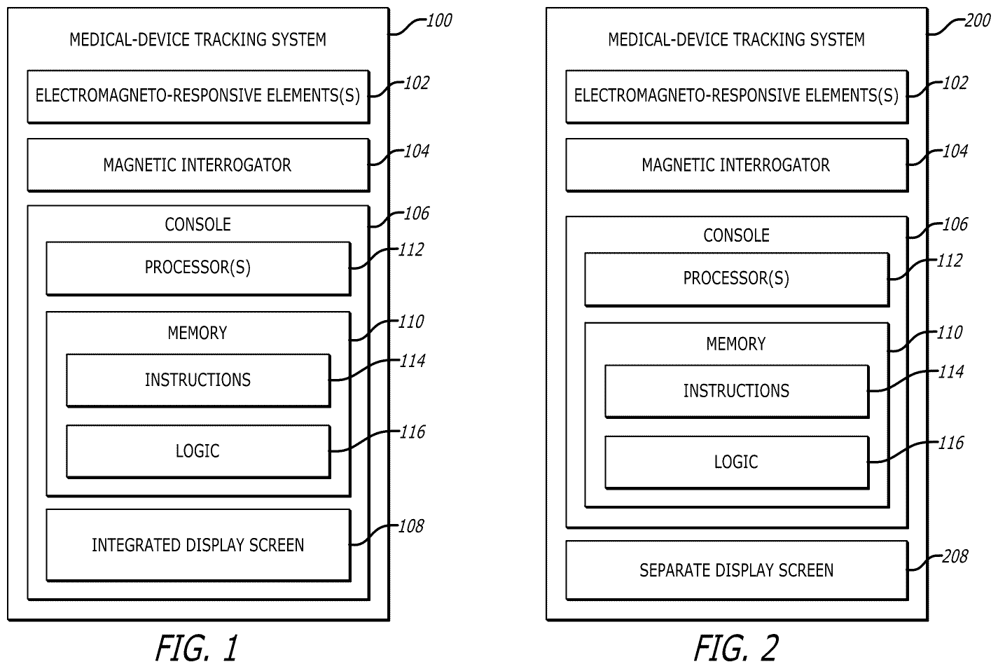

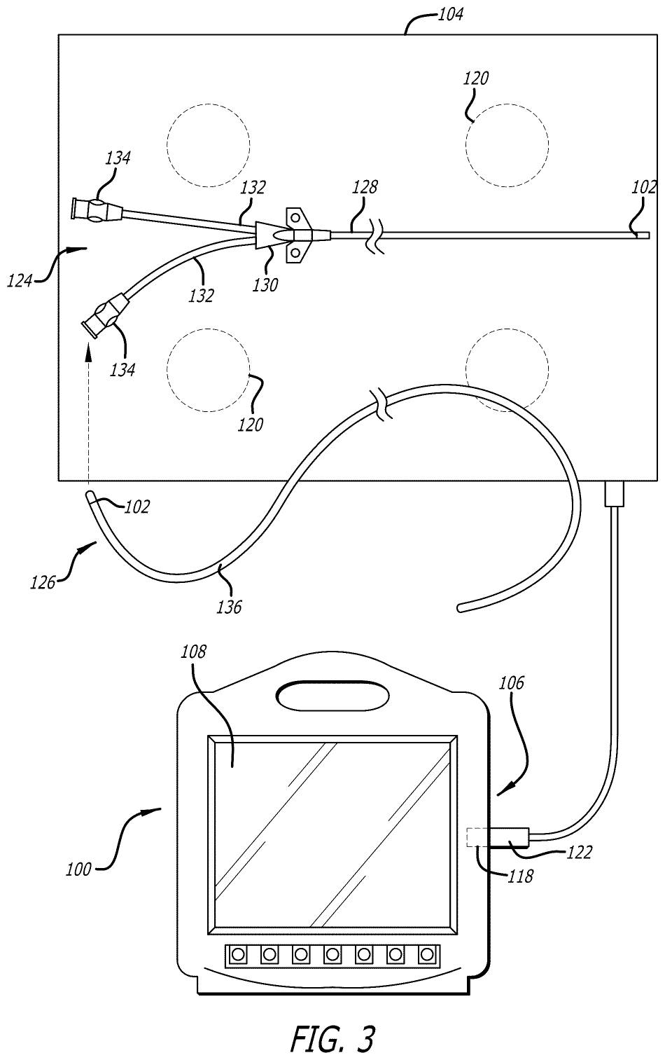

FIG. 1 is a block diagram of a first medical-device navigation system 100 with one or more EMR elements 102 in accordance with some embodiments. FIG. 2 is a block diagram of a second medical-device navigation system 200 with the EMR element(s) 102 in accordance with some embodiments. FIG. 3 illustrates the medical-device navigation system 100 with at least one of the stylet 126 or the catheter 124 as a medical device of the medical-device navigation system 100 including the EMR element(s) 102 in accordance with some embodiments.

As shown, the medical-device navigation system 100 includes the EMR element(s) 102, a stand-alone magnetic interrogator 104, a console 106, and an integrated display screen 108, wherein the integrated display screen 108 is integrated into the console 106. The medical-device navigation system 200 includes the EMR element(s) 102, the magnetic interrogator 104, the console 106, albeit without the integrated display screen 108, and a separate display screen 208 such as that of a stand-alone monitor. However, medical-device navigation systems are not limited to the medical-device navigation system 100 or 200. Indeed, such medical-device navigation systems are examples that convey certain concepts of navigating or placing medical devices with the EMR element(s) 102. With this in mind, description set forth below is primarily provided with respect to the medical-device navigation system 100 for expository expediency, but such description can be extended to the medical-device navigation system 200 and similar systems.

### Consoles

The console 106 has electronic components and circuitry including memory 110 and one or more processors 112. The memory 110 includes executable instructions 114 configured to instantiate medical-device navigation processes upon execution by the processor(s) 112 for navigating medical devices including the EMR element(s) 102 as each medical device or an elongate portion thereof is advanced to a target location. Such medical-device navigation processes are set forth below in more detail with respect to the various modalities of the medical-device navigation system 100 for navigating or placing medical devices. In addition, the console 106 includes logic 116 of various types such as that set forth below with respect to the various modalities of the medical-device navigation system 100 for navigating or placing medical devices.

The console 106 also includes a magnetic-interrogator connector 118. The magnetic-interrogator connector 118 can be configured as a standard power-and-data connector complementary to that of the magnetic interrogator 104 set forth below for operably connecting the magnetic interrogator 104 to the console 106 or disconnecting the magnetic interrogator 104 from the console 106. Advantageously, the console 106 can be configured to automatically instantiate the medical-device navigation processes for navigating or placing medical devices with the EMR element(s) 102 when the magnetic interrogator 104 is connected to the console 106.

### Magnetic Interrogator

FIG. 3 illustrates the magnetic interrogator 104 of the medical-device navigation system 100 in accordance with some embodiments.

The magnetic interrogator 104 is configured to electromagnetically generate an external magnetic field through which medical devices including the EMR element(s) 102 move. In addition, the magnetic interrogator 104 is configured to transduce resonance-based responses of the EMR element(s) 102 to the external magnetic field with a plurality of magnetic sensors 120 having a known relationship to each other. Lastly, the magnetic interrogator 104 is configured to provide response data for the EMR element(s) 102 to the console 106 as each medical device or an elongate portion thereof is moved through the external magnetic field and advanced to a target location.

The magnetic interrogator 104 can include a console connector 122. The console connector 122 can be configured as a standard power-and-data connector complementary to that of the console 106 set forth above for operably connecting the magnetic interrogator 104 to the console 106 or disconnecting the magnetic interrogator 104 from the console 106.

It should be understood that successive locations of the EMR element(s) 102 of each medical device moved through the external magnetic field of the magnetic interrogator 104 are defined in a magnetic interrogator-based coordinate system, which, in turn, is defined by the magnetic sensors 120 of the magnetic interrogator 104 and their known relationship to each other. Notably, such a coordinate system can be converted by the console 106 to a patient-based coordinate system defined in a patient model established by way of one or more imaging techniques including, but not limited to, ultrasound imaging, X-ray imaging, computed tomography (“CT”) scanning, or magnetic resonance imaging (“MRI”). Alternatively, one or more physical features of a patient can be used to proportionally fit a non-specific patient model to the patient including the patient-based coordinate system. In accordance with the patient-based coordinate system, the console 106 can display a graphical representation 123 of a medical device such as the catheter 124 in accordance with its location in a vasculature of the patient like that shown in FIGS. 6A and 6B.

### EMR Elements

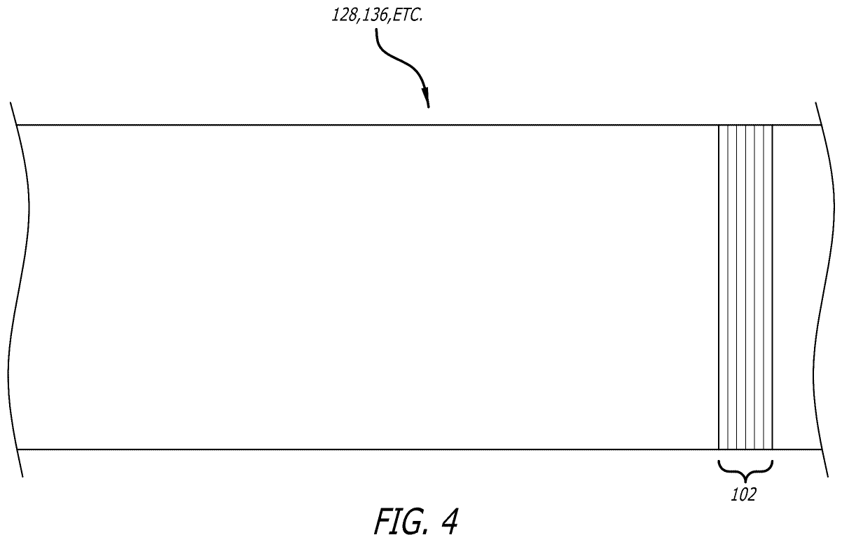

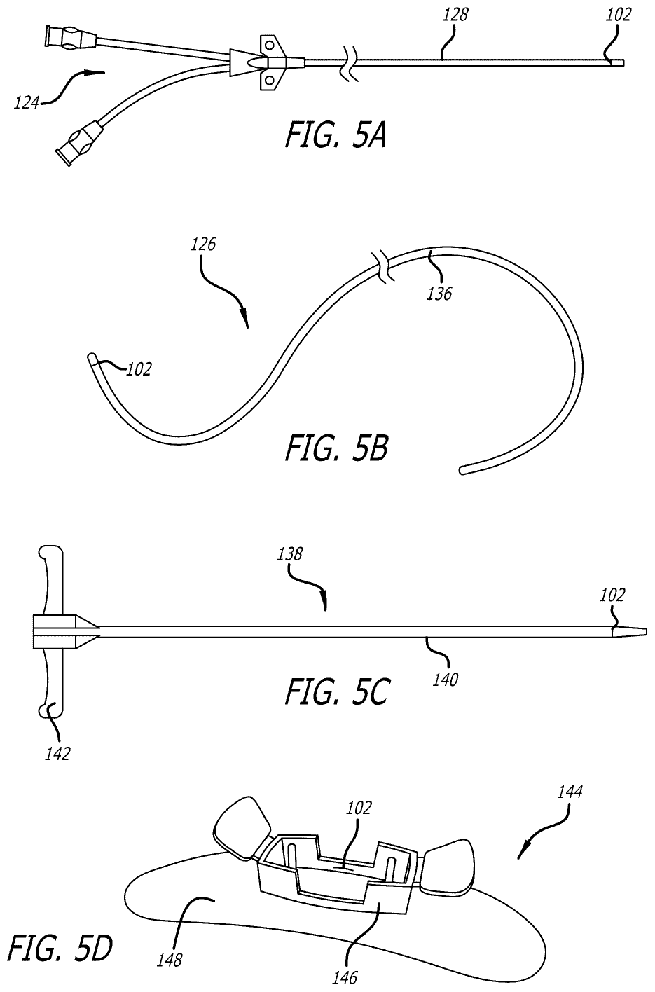

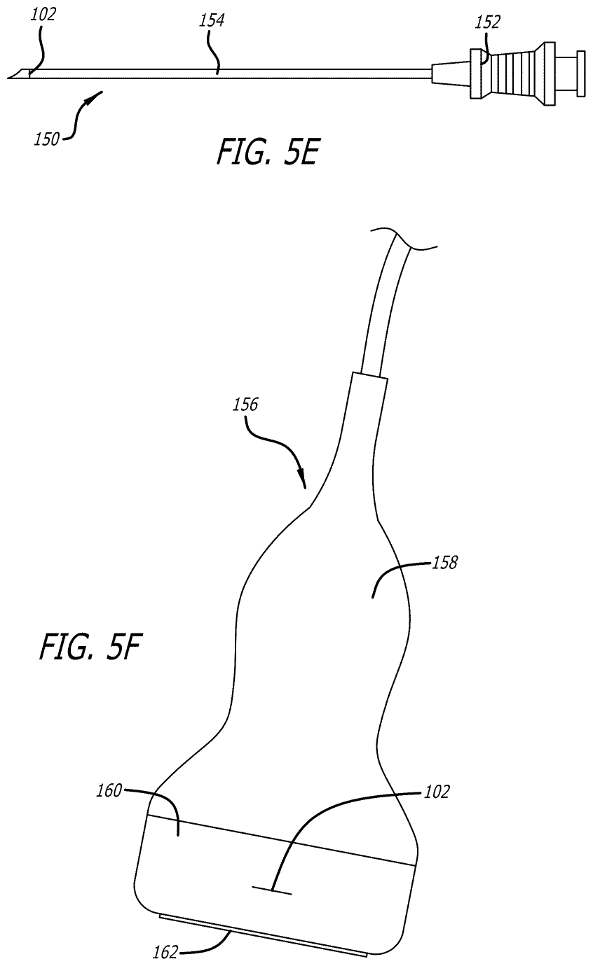

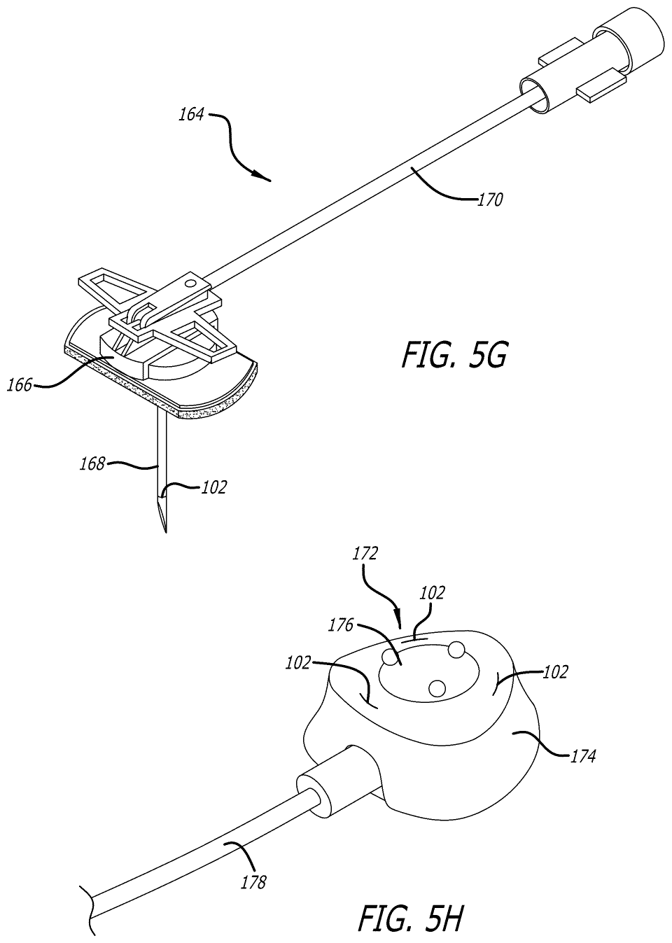

FIG. 4 illustrates a simplified view of a distal end portion of an elongate portion of a medical device including the EMR element(s) 102 in accordance with some embodiments. FIGS. 5A-5H illustrate various medical devices including the EMR element(s) 102 in accordance with some embodiments.

As shown, the EMR element(s) 102 can be in any portion of a medical device disclosed herein; however, the EMR element(s) 102 is(are) typically in an elongate portion of a medical device when the medical device is configured for navigation or placement by way of the medical-device navigation system 100, and, more specifically, the EMR element(s) 102 is(are) typically in a distal end portion of an elongate portion of a medical device when the medical device is configured for navigation or placement by way of the medical-device navigation system 100. Notably, the EMR element(s) 102 can be incorporated within a body of a medical device, such as within the catheter tube 128 of the catheter 124 set forth below, or the EMR element(s) 102 can be incorporated over the body of the medical device, such as over the catheter tube 128 of the catheter 124, optionally, with a coating over the body of the medical device to secure the EMR element(s) 102 over the body as well as provide a smooth surface of the body of the medical device.

The EMR element(s) 102 are passive EMR element(s) 102 in that each EMR element of the EMR element(s) 102 is not internally powered by an internal power source via its corresponding medical device or externally powered by an external power source operably coupled to the medical device. Instead, the EMR element(s) 102 are responsive to the external magnetic field of the magnetic interrogator 104, itself. While embracing some theoretical flexibility, each EMR element of the EMR element(s) 102 can have a natural frequency of vibration that, when matched by the external magnetic field, urges the EMR element 102 to resonate with the external magnetic field in response. Such resonance, in turn, creates local magnetic fields about the EMR element(s) 102 that are detected and transduced by the magnetic sensors 120 of the magnetic interrogator 104.

The EMR element(s) 102 can have sufficient physical and chemical integrity for sterilization by dry heat, moist heat, optionally, in combination with pressure (e.g., by an autoclave), a biocide (e.g., hydrogen peroxide, ethylene oxide, etc.), optionally, in combination with pressure, radiation (e.g., ultraviolet radiation), or a combination thereof when the medical device including the EMR element(s) 102 is sterilized.

### Medical Devices

FIGS. 5A-5H illustrate various medical devices including the EMR element(s) 102 in accordance with some embodiments.

Any medical device that can be navigated or placed by way of the medical-device navigation system 100 or any medical device that can be used to navigate or place another medical device such as the foregoing medical device by way of the medical-device navigation system 100 can include the EMR element(s) 102. And, as set forth above, such EMR element(s) 102 can be in any portion of a medical device; however, the EMR element(s) 102 is(are) typically in an elongate portion of a medical device when the medical device is configured for navigation or placement by way of the medical-device navigation system 100, and, more specifically, the EMR element(s) 102 is(are) typically in a distal end portion of an elongate portion of a medical device when the medical device is configured for navigation or placement by way of the medical-device navigation system 100.

FIG. 5A illustrates a catheter 124 including the EMR element(s) 102 in accordance with some embodiments, and FIG. 5B illustrates a stylet 126 including the EMR element(s) 102 in accordance with some embodiments.

The medical device including the EMR element(s) 102 can be an elongate medical device such as an intravascular medical device selected from the catheter 124, for example, a CVC or PICC, shown in FIG. 5A and the stylet 126 or some other probe shown in FIG. 5B. Notably, if the medical device to be navigated or placed by way of the medical-device navigation system 100 is the catheter 124, the catheter 124 can include the EMR element(s) 102, the stylet 126 can include the EMR element(s) 102, or both the catheter 124 and the stylet 126 can include the EMR element(s) 102 for redundancy or some other function, as set forth below.

With the catheter 124 as an example of the medical device, the catheter 124 can include a catheter tube 128, a catheter hub 130, one or more extension legs 132, and one or more Luer connectors 134 operably and respectively connected in the foregoing order. When such a catheter 124 is multiluminal (e.g., diluminal, triluminal, etc.), the catheter tube 128 includes multiple catheter-tube lumens (e.g., two catheter-tube lumens, three catheter-tube lumens, etc.), the catheter hub 130 is furcated (e.g., bifurcated, trifurcated, etc.) with multiple catheter-hub lumens (e.g., two catheter-hub lumens, three catheter-hub lumens, etc.) correspondingly fluidly connected to the multiple catheter-tube lumens (e.g., the two catheter-tube lumens, the three catheter-tube lumens, etc.), and each extension leg of the two extension legs 132 has an extension-leg lumen fluidly connected to a hub lumen of the multiple catheter-hub lumens (e.g., the two catheter-hub lumens, the three catheter-hub lumens, etc.), thereby providing the multiluminal catheter 124. The EMR element(s) 102 can be incorporated within a distal end portion of the catheter tube 128 of the catheter 124, or the EMR element(s) 102 can be incorporated over the distal end potion of the catheter tube 128, optionally, with a coating over the catheter tube 128 to secure the EMR element(s) 102 over the catheter tube 128 as well as provide a smooth surface of the catheter tube 128.

With the stylet 126 as an example of the medical device, the stylet 126 can be configured to be disposed in a lumen of the catheter 124 such as a primary lumen (also known as a distal lumen) of the catheter 124 such that their distal ends or distal tips are approximately coterminal. The EMR element(s) 102 can be incorporated within a distal end portion of a stylet body 136 of the stylet 126, or the EMR element(s) 102 can be incorporated over the distal end portion of the stylet body 136, optionally, with a coating over the stylet body 136 to secure the EMR element(s) 102 over the stylet body 136 as well as provide a smooth surface of the stylet body 136.

FIG. 5C illustrates a catheter introducer 138 including the EMR element(s) 102 in accordance with some embodiments.

The medical device including the EMR element(s) 102 can be an elongate medical device such as an intravascular medical device including the catheter introducer 138 shown in FIG. 5C. Notably, if the medical device to be navigated or placed by way of the medical-device navigation system 100 is the catheter 124, both the catheter 124 and the catheter introducer 138 can include the EMR element(s) 102, as set forth below.

With the catheter introducer 138 as an example of the medical device, the catheter introducer 138 can be configured with a single lumen to accommodate a catheter tube of a catheter such as that of the foregoing catheter 124 when inserted therethrough. The EMR element(s) 102 can be incorporated within a distal end portion of an introducer sheath 140 of the catheter introducer 138, or the EMR element(s) 102 can be incorporated over the distal end portion of the introducer sheath 140, optionally, with a coating over the introducer sheath 140 to secure the EMR element(s) 102 over the introducer sheath 140 as well as provide a smooth surface of the introducer sheath 140. Notably, the catheter introducer 138 can be splittable, the catheter introducer 138 thereby including a splittable hub 142 over a proximal end portion of a splittable introducer sheath. Any EMR element(s) 102 incorporated within the introducer sheath 140 of the catheter introducer 138 or over the introducer sheath 140 are therefore also configured to split to allow the catheter introducer 138 to be removed from, for example, the catheter 124 upon insertion of the catheter 124 into a vasculature of a patient.

FIG. 5D illustrates a securement device 144 including the EMR element(s) 102 in accordance with some embodiments.

The medical device including the EMR element(s) 102 can be configured to support an elongate medical device, such a medical device including the securement device 144 shown in FIG. 5D. When the securement device 144 is configured to support the catheter 124, the securement device 144 can more specifically be referred to as a catheter securement device. Notably, if the medical device to be navigated or placed by way of the medical-device navigation system 100 is the catheter 124, both the catheter 124 and the securement device 144 can include the EMR element(s) 102, as set forth below.

With the securement device 144 as an example of the medical device, the securement device 144 can be configured with a holder 146 such as a catheter-hub holder for securing an extracorporeal portion of the catheter 124 such as the catheter hub 130 to a surface of a patient such as skin by way of an adhesive pad 148 to which the holder 146 is fixedly attached. The EMR element(s) 102 can be incorporated into the holder 146 or catheter-hub holder of the securement device 144 when the securement device 144 is the catheter securement device.

FIG. 5E illustrates a needle 150 including the EMR element(s) 102 in accordance with some embodiments.

The medical device including the EMR element(s) 102 can be an elongate medical device such as an intravascular medical device including the needle 150 shown in FIG. 5E. Notably, if the medical device to be navigated or placed by way of the medical-device navigation system 100 is the needle 150, both the needle 150 and the ultrasound probe 156 can include the EMR element(s) 102, as set forth below.

With the needle 150 as an example of the medical device, the needle 150 can include a needle hub 152 about a proximal end portion of a needle shaft 154, the needle shaft 154 terminating with a distal tip or needle tip configured for percutaneous insertion into a vasculature of a patient. The EMR element(s) 102 can be incorporated within a distal end portion of the needle shaft 154 of the needle 150 such as within a circumferential groove around the distal end portion of the needle shaft 154. Alternatively, the EMR element(s) 102 can be incorporated over the distal end portion of the needle shaft 154. In either of the foregoing configurations, the needle shaft 154 can have a coating thereover to secure the EMR element(s) 102 to the needle shaft 154 as well as provide a smooth surface of the needle shaft 154.

FIG. 5F illustrates an ultrasound probe 156 including the EMR element(s) 102 in accordance with some embodiments.

The medical device including the EMR element(s) 102 can be configured for use with an elongate medical device, such a medical device including the ultrasound probe 156 shown in FIG. 5F. Indeed, the ultrasound probe 156 can be used to increase a likelihood of first-stick success by way of ultrasound imaging a vasculature of a patient therewith during a percutaneous puncture with the needle 150. Notably, if the medical device to be navigated or placed by way of the medical-device navigation system 100 is the needle 150, both the needle 150 and the ultrasound probe 156 can include the EMR element(s) 102, as set forth below.

With the ultrasound probe 156 as an example of the medical device, the ultrasound probe 156 can include a probe body 158 and a probe head 160 over a distal end portion of the probe body 158, the probe head 160 of the ultrasound probe 156 including a sensor array 162 of ultrasound sensors for the ultrasound imaging. The EMR element(s) 102 can be incorporated into the probe body 158, the probe head 160, as shown, or both the probe body 158 and the probe head 160.

FIG. 5G illustrates a Huber needle 164 including the EMR element(s) 102 in accordance with some embodiments.

The medical device including the EMR element(s) 102 can be an elongate medical device such as an intravascular medical device including the Huber needle 164 shown in FIG. 5G. Notably, if the medical device to be navigated or placed by way of the medical-device navigation system 100 is the Huber needle 164, both the Huber needle 164 and the venous access port 172 can include the EMR element(s) 102, as set forth below.

With the Huber needle 164 as an example of the medical device, the Huber needle 164 can include a needle hub 166 such as a winged needle hub about a proximal end portion of a needle shaft 168, the needle shaft 168 terminating with a distal tip or needle tip configured for percutaneous insertion into the venous access port 172 of a patient. However, it should be understood that the Huber needle 164 need not be that of the infusion set shown in FIG. 5G with the 90-degree needle-shaft-to-needle-hub joint and the extension line 170. Indeed, the Huber needle 164 can be akin to the needle 150 of FIG. 5E, albeit with the angled distal tip of the Huber needle 164. The EMR element(s) 102 can be incorporated within a distal end portion of the needle shaft 168 of the Huber needle 164 such as within a circumferential groove around the distal end portion of the needle shaft 168. Alternatively, the EMR element(s) 102 can be incorporated over the distal end portion of the needle shaft 168. In either of the foregoing configurations, the needle shaft 168 can have a coating thereover to secure the EMR element(s) 102 to the needle shaft 168 as well as provide a smooth surface of the needle shaft 168.

FIG. 5H illustrates a venous access port 172 including the EMR element(s) 102 in accordance with some embodiments.

The medical device including the EMR element(s) 102 can be an intravascular medical device including the venous access port 172 shown in FIG. 5H. Notably, if the medical device to be navigated or placed by way of the medical-device navigation system 100 is the Huber needle 164, both the Huber needle 164 and the venous access port 172 can include the EMR element(s) 102, as set forth below.

With the venous access port 172 as an example of the medical device, the venous access port 172 can include a housing 174, a reservoir within the housing 174 (discernable by the septum 176), and a septum 176 covering the reservoir for repeated access to the reservoir by way of the Huber needle 164 for delivering medication or the like to a patient by way of a catheter tube 178 placed in an SVC of a patient. The EMR element(s) 102 can be incorporated in the housing 174 of the venous access port 172 such as around the reservoir or the septum 176 covering the reservoir. Additionally or alternatively, the EMR element(s) 102 can be incorporated within a distal end portion of the catheter tube 178 of the venous access port 172, or the EMR element(s) 102 can be incorporated over the distal end potion of the catheter tube 178, optionally, with a coating over the catheter tube 178 to secure the EMR element(s) 102 over the catheter tube 178 as well as provide a smooth surface of the catheter tube 178.

### Modalities

As set forth above, modalities of the medical-device navigation system 100 can be broadly divided among the following two modalities: 1) One or more EMR elements 102, for example, a single EMR element 102, of a medical device is used for navigating or placing that medical device as it is moved through an external magnetic field to a target location. 2) Two or more EMR elements 102, for example, a pair of EMR elements 102, distributed between two or more medical devices, are used together for navigating or placing one of the two or more medical devices as that medical device is moved through the external magnetic field to a target location. Notably, the two or more EMR elements 102 can be distributed between two or more medical devices in any combination so long as two medical devices of the two or more medical devices have at least one EMR element 102 apiece.

FIGS. 6A and 6B illustrate the first modality of the medical-device navigation system 100 in accordance with some embodiments.

As to the first modality of the medical-device navigation system 100, wherein the one or more EMR elements 102, for example, the single EMR element 102, of a medical device is used for navigating or placing that medical device as it is moved through an external magnetic field to a target location, the medical-device navigation processes include various processes selected from at least a data-acquisition process, a triangulation process, and a plotting process.

The data-acquisition process utilizes data-acquisition logic for acquiring the response data from the magnetic interrogator 104 over time and writing the response data to the memory 110 of the console 106. As set forth above, the magnetic interrogator 104 is configured to provide such response data to the console 106 as the medical device or elongate portion thereof including the EMR element(s) 102 is moved through the external magnetic field generated by the magnetic interrogator 104 and advanced to the target location.

The triangulation process utilizes triangulation logic with the response data for triangulating the EMR element(s) 102 with respect to each of the magnetic sensors 120 over time and writing 3D location data to the memory 110 of the console 106. The 3D location data is set in the magnetic interrogator-based coordinate system set forth above, which coordinate system is defined by the magnetic sensors 120 of the magnetic interrogator 104 and their known relationship to each other.

The plotting process utilizes plotting logic with the 3D location data for plotting locations of the EMR element(s) 102 on the display screen 108 or 208 associated with the console 106 over time as the elongate portion of the medical device is advanced through the external magnetic field to the target location. Notably, successive locations of the EMR element(s) 102 en route to the target location can be concatenated on the display as shown in FIGS. 6A and 6B, thereby providing a tracking path for at least the elongate portion of the medical device.

FIGS. 6A and 6B also illustrate the medical-device navigation system 100 with the stylet 126 and catheter 124 being advanced to a target location in a body of a patient in accordance with some embodiments.

In such embodiments, the target location can be an SVC of the patient. In accordance with the first modality of the medical-device navigation system 100, the plotting process further plots the location of the EMR element(s) 102 over a patient avatar 180 on the display screen 108 or 208 associated the console 106 in real-time as the elongate portion of the medical device including the EMR element(s) 102 is advanced through the body of the patient to the SVC. In an example, when the medical device is a CVC, the plotting process further plots the location of the EMR element(s) 102 over the patient avatar 180 on the display screen 108 or 208 as the distal end portion of the CVC including the EMR element(s) 102 is advanced through a vasculature of the patient including a right internal jugular vein, a right brachiocephalic vein, and into the SVC. In another example, when the medical device is a PICC, the plotting process further plots the location of the EMR element(s) 102 over the patient avatar 180 on the display screen 108 or 208 as the distal end portion of the PICC including the EMR element(s) 102 is advanced through the vasculature of the patient including a right basilic vein, a right axillary vein, a right subclavian vein, a right brachiocephalic vein, and into the SVC. That said, the medical device with the EMR element(s) 102 can alternatively be the stylet 126 disposed in a primary lumen of the CVC or PICC such that their distal ends are approximately coterminal. Indeed, in such embodiments, the plotting process further plots the location of the EMR element(s) 102 over the patient avatar 180 on the display screen 108 or 208 as the distal end portion of the stylet 126 including the EMR element(s) 102 is advanced through the foregoing vasculature of the patient.

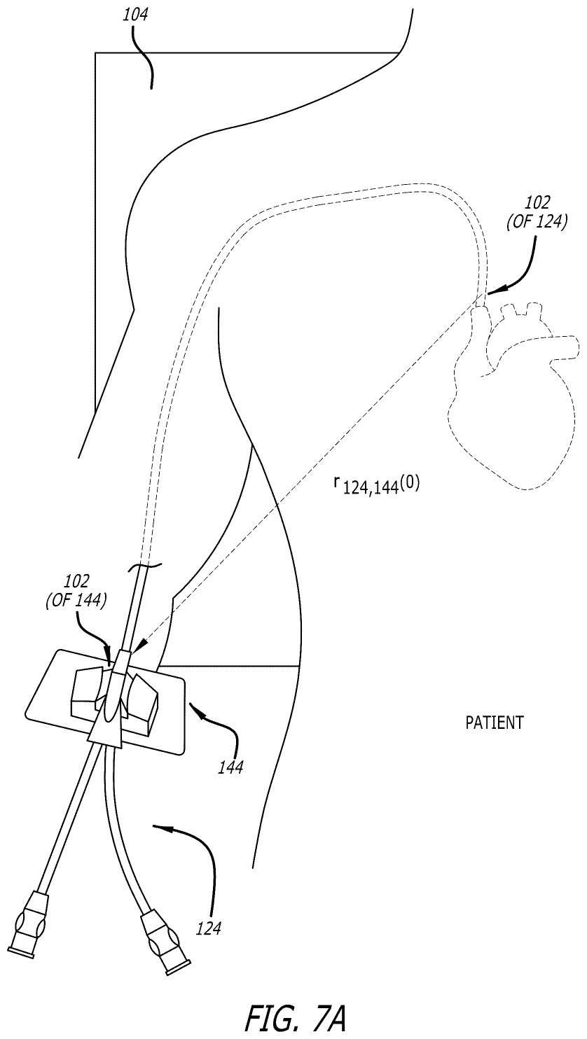

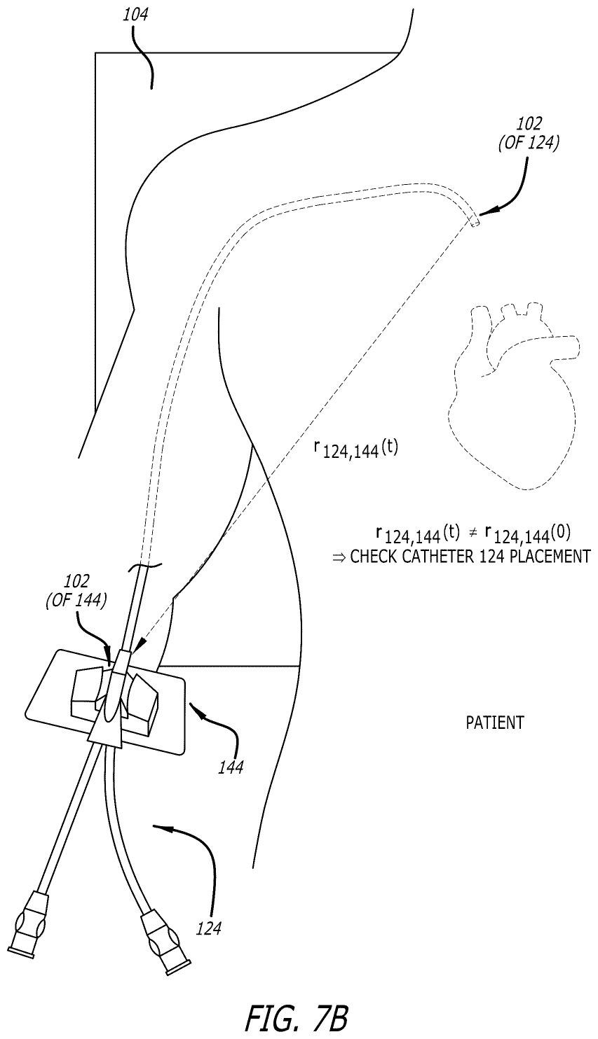

FIGS. 7A, 7B, 8A, 8B, 9A, 9B, and 10A-10C illustrate the second modality of the medical-device navigation system 100 in accordance with some embodiments.

As to the second modality of the medical-device navigation system 100, wherein the two or more EMR elements 102, for example, the pair of EMR elements 102 such as a first EMR element 102 and a second EMR element 102, distributed between two or more medical devices, respectively, are used together for navigating or placing one of the two or more medical devices as that medical device is moved through the external magnetic field to a target location, the medical-device navigation processes include various processes selected from at least a data-acquisition process, a triangulation process, a vectoring process, a vector-analysis process, and a displaying process. For expository expediency, the medical device of the two or more medical devices that is moved through the external magnetic field to the target location is referred to as a navigable medical device below. Any other medical device of the two or more medical devices is referred to as a stationary medical device below.

Like that set forth above, the data-acquisition process utilizes data-acquisition logic for acquiring the response data from the magnetic interrogator 104 over time and writing the response data to the memory 110 of the console 106. The magnetic interrogator 104 is configured to provide such response data to the console 106 as the navigable medical device or elongate portion thereof including at least the first EMR element 102 is moved through the external magnetic field generated by the magnetic interrogator 104 and advanced to the target location. However, the magnetic interrogator 104 is also configured to provide such response data to the console 106 as the stationary medical device including at least the second EMR element 102 is held substantially stationary in the external magnetic field generated by the magnetic interrogator 104.

Like that set forth above, the triangulation process utilizes triangulation logic with the response data for triangulating at least the first and second EMR elements 102 with respect to each of the magnetic sensors 120 over time and writing 3D location data to the memory 110 of the console 106. Again, the 3D location data is set in the magnetic interrogator-based coordinate system set forth above, which coordinate system is defined by the magnetic sensors 120 of the magnetic interrogator 104 and their known relationship to each other.

The vectoring process utilizes vectoring logic with the 3D location data for at least the first and second EMR elements 102 over time to determine absolute location and velocity vectors for the first and second EMR elements 102 in the coordinate system established by the magnetic sensors 120. If needed, the vectoring process utilizes the vectoring logic with the foregoing 3D location data, the absolute location and velocity vectors for the first and second EMR elements 102, or some combination thereof to determine relative location and velocity vectors between the first and second EMR elements 102 in the coordinate system established by the magnetic sensors 120. Lastly, the vectoring process writes any determined vectors of the foregoing vectors to the memory 110 of the console 106.

The vector-analysis process utilizes vector-analysis logic with some combination of the foregoing vectors for determining any movement of the first EMR element 102 in the elongate portion of the navigable medical device through the external magnetic field with respect to the second EMR element 102 in the stationary medical device, and, ultimately, the target location, particularly, when the target location is the stationary medical device. Indeed, the vector-analysis process can utilize vector-analysis logic with some combination of the foregoing vectors for determining their dot products over time, which indicate whether the first EMR element 102 in the navigable medical device is moving toward or away from the second EMR element 102 in the stationary medical device. For example, at any given time, the dot product of a) the relative location vector between the first EMR element 102 in the navigable medical device and the second EMR element 102 in the stationary medical device and b) the relative velocity vector between the first and second EMR elements 102 can indicate whether the first EMR element 102 is moving toward or away from the second EMR element 102. When the foregoing dot product is negative, the first EMR element 102 in the navigable medical device is moving toward the second EMR element 102 in the stationary medical device. When the foregoing dot product is positive, the first EMR element 102 in the navigable medical device is moving away from the second EMR element 102 in the stationary medical device. And when the foregoing dot product is zero, the first EMR element 102 in the navigable medical device is neither moving toward nor away from the second EMR element 102 in the stationary medical device.

FIGS. 7A and 7B also illustrate the medical-device navigation system 100 with the catheter 124 being displaced from a target location in a body of a patient as determined by reference to the securement device 144 in accordance with some embodiments.

In such embodiments, the target location can be an SVC of the patient, the navigable medical device can be a CVC or PICC, with the latter shown, and the stationary medical device can be the securement device 144 for securing an extracorporeal portion of the CVC or PICC to the patient. Like that set forth above, the first EMR element 102 is in the distal end portion of the catheter tube 128 of the CVC or PICC such as about a distal tip of the catheter tube 128, and the second EMR element 102 is incorporated into the holder 146 or catheter-hub holder of the securement device 144.

In accordance with the second modality of the medical-device navigation system 100, the vector-analysis process of determining any movement of the first EMR element 102 through the external magnetic field with respect to the target location includes determining any displacement of the distal tip of the CVC or PICC from the SVC by way of the location or velocity vectors for the first EMR element 102 in the distal tip of the CVC or PICC and the second EMR element 102 in the holder 146 or catheter-hub holder of the securement device 144. This is simply illustrated between FIGS. 7A and 7B, wherein the relative location vector from the first EMR element 102 of the CVC or PICC to the second EMR element 102 of the securement device 144 at time t, namely, r_{124,144}(t), is not equal to r_{124,144}(0), the relative location vector from the first EMR element 102 of the CVC or PICC to the second EMR element 102 of the securement device 144 at time 0 immediately following placement of the CVC or PICC, thereby indicting displacement. Further, like that shown in FIGS. 6A and 6B, the displaying process includes displaying any displacement of the distal tip of the CVC or PICC from the SVC with the graphical representation 123 of the elongate portion of the CVC or PICC over the patient avatar 180 on the display screen 108 or 208 associated with the console 106.

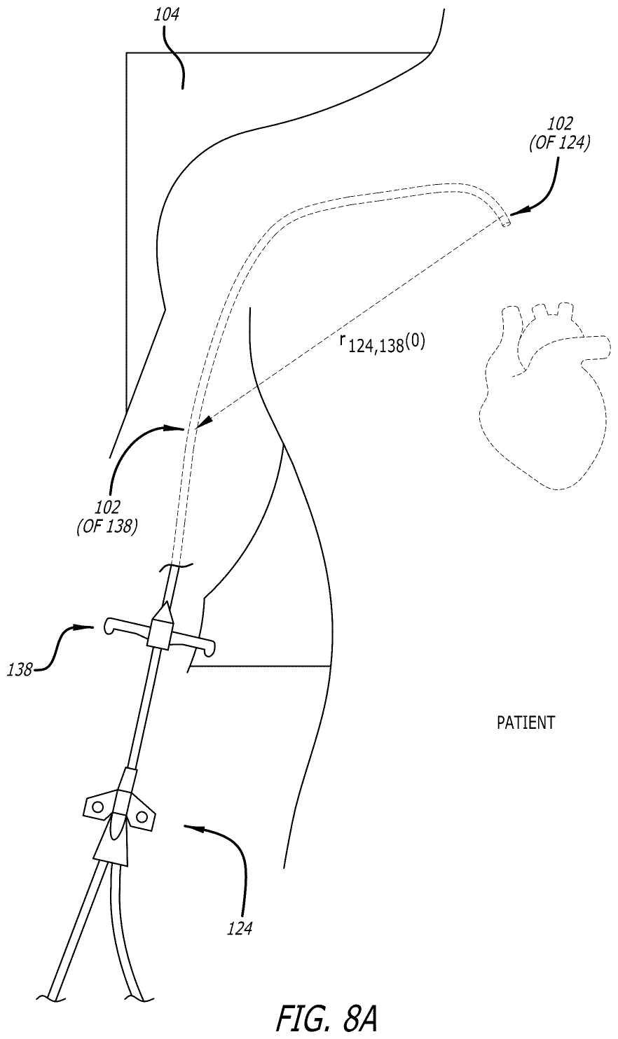

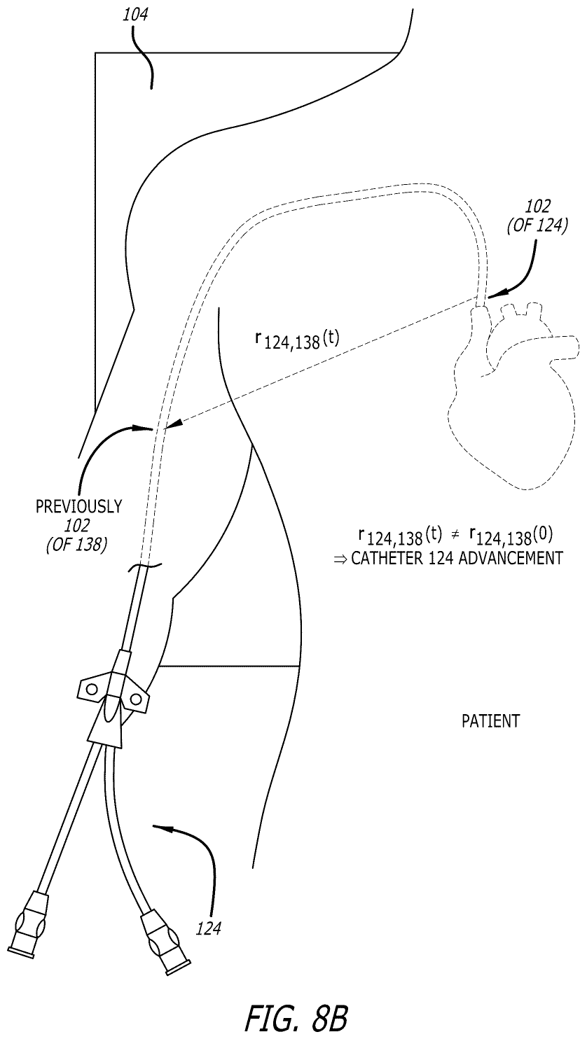

FIGS. 8A and 8B also illustrate the medical-device navigation system 100 with the catheter 124 being advanced to a target location in a body of a patient as determined by reference to an initial location of the catheter introducer 138 in accordance with some embodiments.

In such embodiments, the target location can be an SVC of the patient, the navigable medical device can be a CVC or PICC, with the latter shown, and the stationary medical device can be the catheter introducer 138 for introducing the elongate portion of the CVC or PICC to a vasculature of the patient. Like that set forth above, the first EMR element 102 is in the distal end portion of the catheter tube 128 of the CVC or PICC such as about the distal tip of the catheter tube 128, and the second EMR element 102 is incorporated into a distal tip of the catheter introducer 138.

In accordance with the second modality of the medical-device navigation system 100, the vector-analysis process of determining any movement of the first EMR element 102 through the external magnetic field with respect to the target location includes determining advancement of the distal tip of the CVC or PICC toward the SVC by way of the location or velocity vectors for the first EMR element 102 in the distal tip of the CVC or PICC and the second EMR element 102 in the distal tip of the catheter introducer 138. The location or velocity vectors for the first EMR element 102 of the CVC or PICC and the second EMR element 102 of the catheter introducer 138 need not be with respect to a common time. Indeed, the vector-analysis process can utilize the location or velocity vectors for the second EMR element 102 of the catheter introducer 138 at a time prior to removing the catheter introducer 138 from the patient such as those previously written to the memory 110 of the console 106. The vector-analysis process can further utilize the location or velocity vectors for the first EMR element 102 of the CVC or PICC while advancing the distal tip of the CVC or PICC toward the SVC at a time after removing the catheter introducer 138 from the patient such as those instantly being written to the memory 110 of the console 106. This is simply illustrated between FIGS. 8A and 8B, wherein the relative location vector at time 0 for the first EMR element 102 of the CVC or PICC to the second EMR element 102 of the catheter introducer 138 in its current location, namely, r_{124,138}(0), is shorter than r_{124,138}(t), the relative location vector at time t for the first EMR element 102 of the CVC or PICC to the second EMR element 102 of the catheter introducer 138 in its previous location, thereby indicting advancement of the CVC or PICC, as expected, in accordance with the CVC or PICC doubling back down the right brachiocephalic vein. Further, like that shown in FIGS. 6A and 6B, the displaying process includes displaying advancement of the distal tip of the CVC or PICC toward the SVC with the graphical representation 123 of the elongate portion of the CVC or PICC over the patient avatar 180 on the display screen 108 or 208 associated with the console 106.

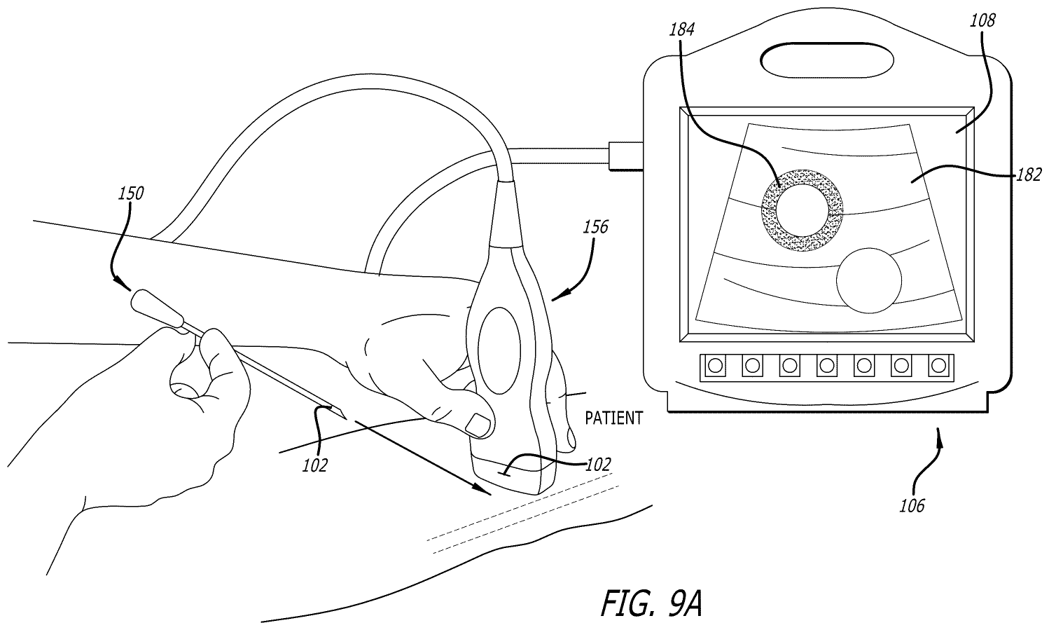

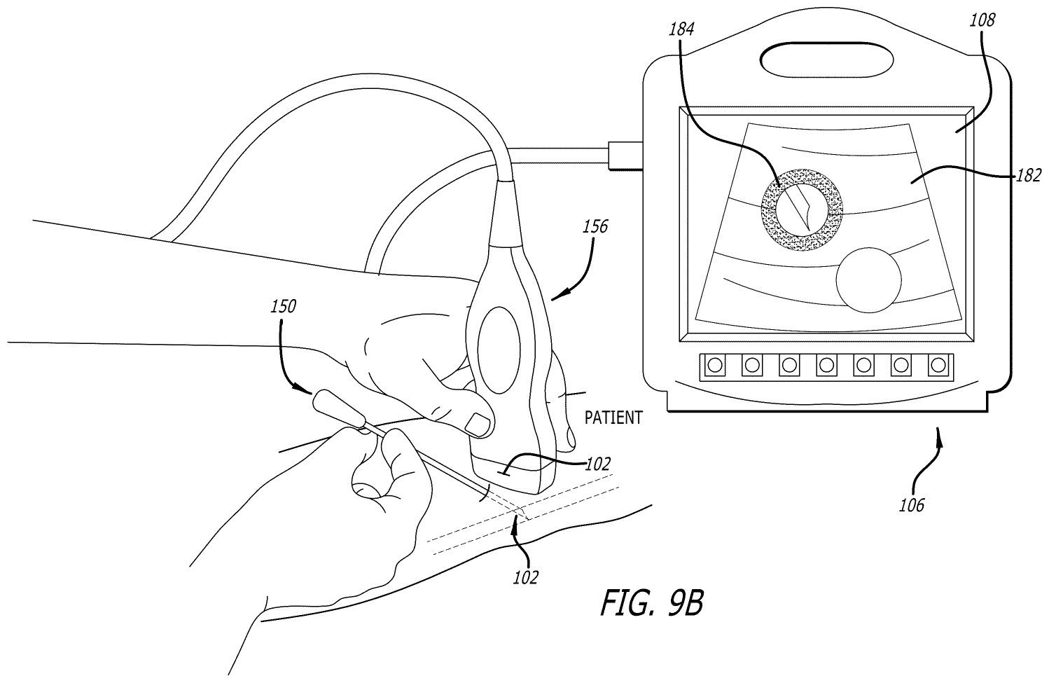

FIGS. 9A and 9B also illustrate the medical-device navigation system 100 with the needle 150 being advanced to a target location in a body of a patient as determined by reference to a location of the ultrasound probe 156 while ultrasound imaging in accordance with some embodiments.

In such embodiments, the target location can be a blood vessel in a vasculature of the patient, the navigable medical device can be the needle 150, and the stationary medical device can be the ultrasound probe 156 for ultrasound imaging the vasculature of the patient. Like that set forth above, the first EMR element 102 is in the distal end portion of the needle shaft 154 of the needle 150 such as about the distal tip of the needle shaft 154, and the second EMR element 102 is incorporated into the probe head 160 of the ultrasound probe 156.

In accordance with the second modality of the medical-device navigation system 100, the vector-analysis process of determining any movement of the first EMR element 102 through the external magnetic field with respect to the target location includes determining advancement of the distal tip of the needle 150 toward the blood vessel by way of the location or velocity vectors for the first EMR element 102 in the distal tip of the needle 150, the second EMR element 102 in the probe head 160 of the ultrasound probe 156, and a triangulated location of the blood vessel below the probe head 160 of the ultrasound probe 156 as determined by the ultrasound imaging. Further, like that shown in FIGS. 9A and 9B, the displaying process includes displaying an ultrasound image 182 on the display screen 108 or 208 associated with the console 106 while advancing the distal tip of the needle 150 toward the blood vessel below the probe head 160 of the ultrasound probe 156. Notably, a blood-vessel segment of the ultrasound image 182 corresponding to the blood vessel in the target location below the probe head 160 of the ultrasound probe 156 can be visually indicted, for example, with another graphical representation 184 such as an on-screen icon or effect, in the ultrasound image 182 when the needle 150 is aligned therewith.

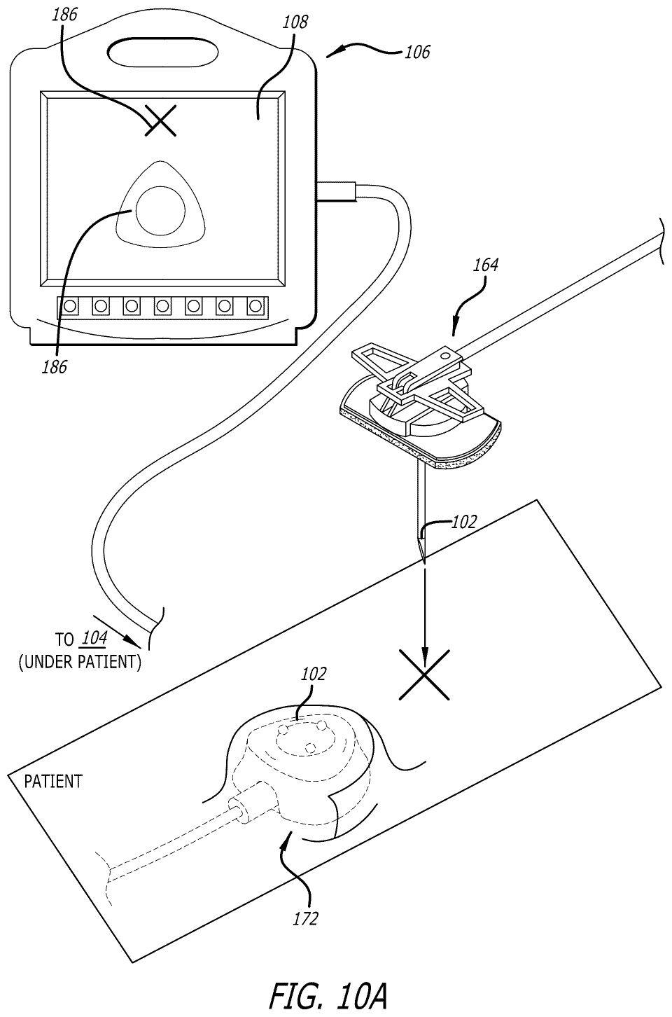

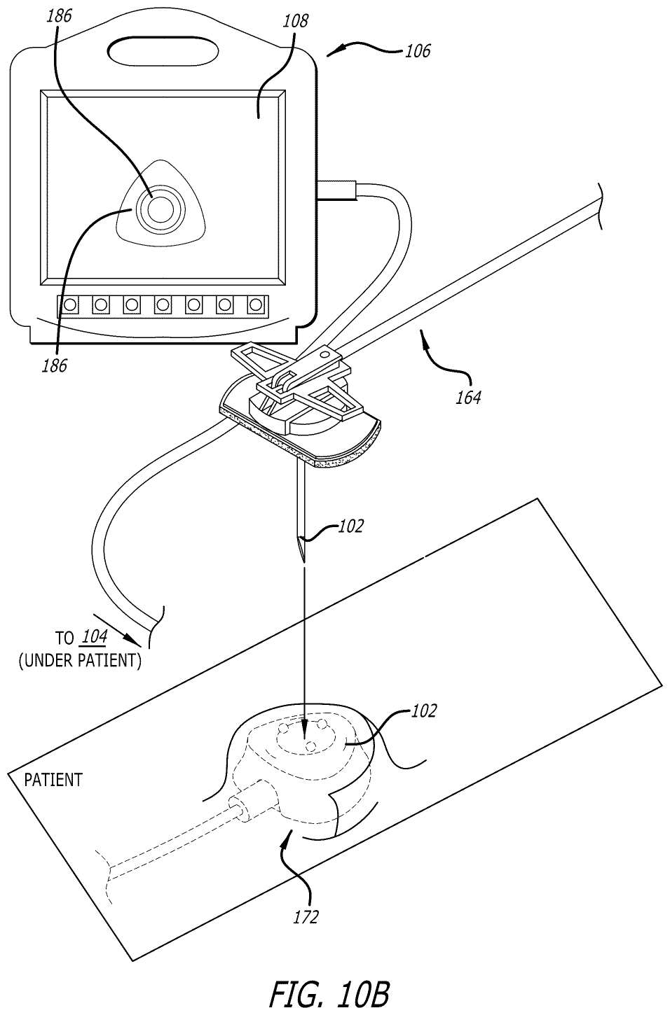

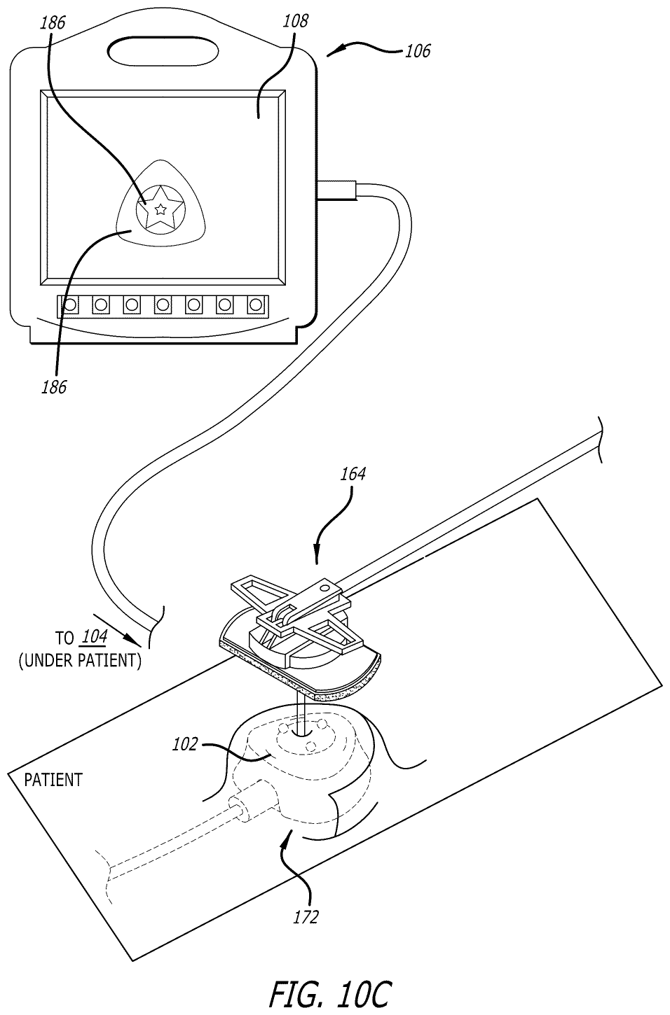

FIGS. 10A-10C also illustrate the medical-device navigation system 100 with the Huber needle 164 being advanced to the venous access port 172 as a target location in a body of a patient in accordance with some embodiments.

In such embodiments, the navigable medical device can be the Huber needle 164, the stationary medical device can be the venous access device, and the target location can also be the venous access device such as the reservoir thereof, which is covered by the septum 176. Like that set forth above, the first EMR element 102 is in the distal end portion of the needle shaft 168 of the Huber needle 164 such as about the distal tip of the needle shaft 168, and the second EMR element 102, optionally, with one or more additional EMR elements 102, is incorporated into the venous access port 172 such as in the housing 174 around the septum 176 thereof.

In accordance with the second modality of the medical-device navigation system 100, the vector-analysis process of determining any movement of the first EMR element 102 through the external magnetic field with respect to the target location includes determining advancement of the distal tip of the Huber needle 164 toward the septum 176 of the venous access port 172 by way of the location or velocity vectors for the first EMR element 102 in the distal tip of the Huber needle 164, the second EMR element 102 in the venous access port 172 around the septum 176, and, optionally, the one or more additional EMR elements 102 in the venous access port 172 around the septum 176. Further, like that shown in FIGS. 10A-10C, the displaying process includes displaying advancement of the distal tip of the Huber needle 164 toward the septum 176 of the venous access port 172 with graphical representations 186 of the venous access port 172 and the distal tip of the Huber needle 164 on the display screen 108 or 208 associated with the console 106. Indeed, by way of example, the graphical representation 186 of the venous access port 172 can be a simple on-screen drawing illustrating a top view of the venous access port 172, and the graphical representation 186 of the distal tip of the Huber needle 164 can be an icon that changes in accordance with the distal tip of the Huber needle 164 being out of alignment with the septum 176 of the venous access port 172 such as by a simple ‘X,’ in alignment with the septum 176 of the venous access port 172 such as by a simple ‘O,’ or accessing the reservoir under the septum 176 of the venous access port 172 with a ‘☆.’ However, it should be understood that displaying advancement of the distal tip of the Huber needle 164 toward the septum 176 of the venous access port 172 is not limited to the foregoing graphical representations 186 of the venous access port 172 and the distal tip of the Huber needle 164 on the display screen 108 or 208 associated with the console 106, as various visual indicators can be used to the same effect.

### Methods

Methods of the medical-device navigation system 100 or any medical devices disclosed herein include at least methods of the medical-device navigation system 100, itself, or methods of using the medical-device navigation system 100 or medical devices thereof or associated therewith. Such methods can be discerned from that set forth above regarding functions of the medical-device navigation system 100, functions of the medical devices, uses of the medical-device navigation system 100, uses of the medical devices, or some combination above.

The medical-device navigation system 100 and methods are not limited to the structural configurations and modalities set forth above. Indeed, the structural configuration of the medical-device navigation system 100 can vary, as needed, to provide any one or more modalities set forth above in combination with one or more of those disclosed in U.S. Pat. Nos. 8,388,541; 8,781,555; 8,849,382; 9,636,031; 9,649,048; or some combination thereof, each of which is incorporated by reference in its entirety into this application.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

What is claimed is:

1. A medical-device navigation system comprising:
an electromagnetoresponsive (“EMR”) element in an elongate portion of a medical device, the EMR element responsive to an external magnetic field;
a magnetic interrogator configured to:
generate the external magnetic field;
transduce resonance-based responses of the EMR element to the external magnetic field with a plurality of magnetic sensors; and
provide response data for the EMR element as the elongate portion of the medical device including the EMR element moves through the external magnetic field; and
a console with electronic components and circuitry including memory and one or more processors, the memory including executable instructions configured to instantiate medical-device navigation processes upon execution by the processor(s) for navigating the elongate portion of the medical device including the EMR element as it is advanced to a target location, and the medical-device navigation processes including:
a data-acquisition process that utilizes data-acquisition logic for acquiring the response data from the magnetic interrogator over time and writing the response data to the memory of the console;
a triangulation process that utilizes triangulation logic with the response data for triangulating the EMR element with respect to each of the magnetic sensors over time and writing three-dimensional (“3D”) location data to the memory of the console, the 3D location data set in a coordinate system established by the magnetic sensors; and
a plotting process that utilizes plotting logic with the 3D location data for plotting a location of the EMR element on a display screen of the console over time as the elongate portion of the medical device is advanced through the external magnetic field to the target location.

2. The medical-device navigation system of claim 1, wherein the target location is in a body of a patient, the plotting process further plotting the location of the EMR element over a patient avatar on the display screen of the console in real-time as the elongate portion of the medical device including the EMR element is advanced through the body of the patient to the target location.

3. The medical-device navigation system of claim 2, wherein the medical device is a central venous catheter (“CVC”), the target location is a superior vena cava (“SVC”), and the plotting process further plots the location of the EMR element over the patient avatar on the display screen of the console over time as the elongate portion of the medical device including the EMR element is advanced through a vasculature of the patient including a right internal jugular vein, a right brachiocephalic vein, and into the SVC.

4. The medical-device navigation system of claim 2, wherein the medical device is a stylet configured to be disposed in a primary lumen of a central venous catheter (“CVC”) such that their distal ends are approximately coterminal, the target location is a superior vena cava (“SVC”), and the plotting process further plots the location of the EMR element over the patient avatar on the display screen of the console over time as the elongate portion of the medical device including the EMR element is advanced through a vasculature of the patient including a right internal jugular vein, a right brachiocephalic vein, and into the SVC.

5. The medical-device navigation system of claim 2, wherein the medical device is a peripherally inserted central catheter (“PICC”), the target location is a superior vena cava (“SVC”), and the plotting process further plots the location of the EMR element over the patient avatar on the display screen of the console over time as the elongate portion of the medical device including the EMR element is advanced through a vasculature of the patient including a right basilic vein, a right axillary vein, a right subclavian vein, a right brachiocephalic vein, and into the SVC.

6. The medical-device navigation system of claim 2, wherein the medical device is a stylet configured to be disposed in a primary lumen of a peripherally inserted central catheter (“PICC”) such that their distal ends are approximately coterminal, the target location is a superior vena cava (“SVC”), and the plotting process further plots the location of the EMR element over the patient avatar on the display screen of the console over time as the elongate portion of the medical device including the EMR element is advanced through a vasculature of the patient including a right basilic vein, a right axillary vein, a right subclavian vein, a right brachiocephalic vein, and into the SVC.

7. A medical-device navigation system comprising:
a pair of electromagnetoresponsive (“EMR”) elements responsive to an external magnetic field distributed between a pair of medical devices, a first EMR element in an elongate portion of a first medical device, and a second EMR element in any portion of a second medical device;
a magnetic interrogator configured to:
generate the external magnetic field;
transduce resonance-based responses of the EMR elements to the external magnetic field with a plurality of magnetic sensors; and
provide response data for the EMR elements as the elongate portion of the first medical device including the first EMR element moves through the external magnetic field; and
a console with electronic components and circuitry including memory and one or more processors, the memory including executable instructions configured to instantiate medical-device navigation processes upon execution by the processor(s) for navigating the elongate portion of the first medical device including the first EMR element as it is advanced to a target location, and the medical-device navigation processes including:
a data-acquisition process that utilizes data-acquisition logic for acquiring the response data from the magnetic interrogator over time and writing the response data to the memory of the console;
a triangulation process that utilizes triangulation logic with the response data for triangulating the EMR elements with respect to each of the magnetic sensors over time and writing three-dimensional (“3D”) location data to the memory of the console, the 3D location data set in a coordinate system established by the magnetic sensors;
a vectoring process that utilizes vectoring logic with the 3D location data for determining absolute location and velocity vectors for the EMR elements in the coordinate system established by the magnetic sensors as well as relative location and velocity vectors between the first EMR element and the second EMR element therefrom and writing such vectors to the memory of the console; and
a vector-analysis process that utilizes vector-analysis logic with some combination of the vectors for determining any movement of the first EMR element in the elongate portion of the first medical device through the external magnetic field with respect to the target location.

8. The medical-device navigation system of claim 7, wherein the first medical device is a central venous catheter (“CVC”) or peripherally inserted central catheter (“PICC”) with the first EMR element about a distal tip of a catheter tube of the CVC or PICC, the second medical device is a securement device for securing an extracorporeal portion of the CVC or PICC to a surface of a patient, the second EMR element incorporated into a catheter-hub holder of the securement device, and the target location is a superior vena cava (“SVC”) in a body of the patient.

9. The medical-device navigation system of claim 8, wherein determining any movement of the first EMR through the external magnetic field with respect to the target location includes determining any displacement of the distal tip of the CVC or PICC from the SVC by way of the relative location or velocity vectors between the first EMR element in the distal tip of the CVC or PICC and the second EMR element in the catheter-hub holder of the securement device.

10. The medical-device navigation system of claim 9, the medical-device navigation processes further including displaying any displacement of the distal tip of the CVC or PICC from the SVC with a graphical representation of the elongate portion of the CVC or PICC over a patient avatar on a display screen of the console.

11. The medical-device navigation system of claim 7, wherein the first medical device is a central venous catheter (“CVC”) or peripherally inserted central catheter (“PICC”) with the first EMR element about a distal tip of a catheter tube of the CVC or PICC, the second medical device is a catheter introducer for introducing the elongate portion of the CVC or PICC to a vasculature of a patient, the second EMR element incorporated into a distal tip of the catheter introducer, and the target location is a superior vena cava (“SVC”) in a body of the patient.

12. The medical-device navigation system of claim 11, wherein determining any movement of the first EMR element through the external magnetic field with respect to the target location includes determining advancement of the distal tip of the CVC or PICC toward the SVC by way of the relative location or velocity vectors between the first EMR element in the distal tip of the CVC or PICC and the second EMR element in the distal tip of the catheter introducer.

13. The medical-device navigation system of claim 12, wherein the relative location or velocity vectors between the first EMR element in the distal tip of the CVC or PICC and the second EMR element in the distal tip of the catheter introducer need not be with respect to a common time, the vector-analysis process utilizing the absolute location or velocity vectors for the second EMR element in the distal tip of the catheter introducer at a time prior to removing the introducer catheter from the patient, and the vector-analysis process utilizing the absolute location or velocity vectors for the first EMR element in the distal tip of the CVC or PICC while advancing the distal tip of the CVC or PICC toward the SVC at a time after removing the introducer catheter from the patient.

14. The medical-device navigation system of claim 12, the medical-device navigation processes further including displaying advancement of the distal tip of the CVC or PICC toward the SVC with a graphical representation of the elongate portion of the CVC or PICC over a patient avatar on a display screen of the console.

15. The medical-device navigation system of claim 7, wherein the first medical device is a needle with the first EMR element about a distal tip of a needle shaft of the needle, the second medical device is an ultrasound probe for ultrasound imaging a vasculature of a patient, the second EMR element incorporated into a probe head of the ultrasound probe, and the target location is a blood vessel in the vasculature of the patient.

16. The medical-device navigation system of claim 15, wherein determining any movement of the first EMR through the external magnetic field with respect to the target location includes determining advancement of the distal tip of the needle toward the blood vessel by way of the relative location or velocity vectors between the first EMR element in the distal tip of the needle, the second EMR element in the probe head of the ultrasound probe, and a triangulated location of the blood vessel below the probe head of the ultrasound probe determined by the ultrasound imaging.

17. The medical-device navigation system of claim 16, the medical-device navigation processes further including displaying an ultrasound image on a display screen of the console while advancing the distal tip of the needle toward the blood vessel below the probe head of the ultrasound probe, a blood-vessel segment of the ultrasound image corresponding to the blood vessel below the probe head of the ultrasound probe visually indicted in the ultrasound image when the needle is aligned therewith.

18. The medical-device navigation system of claim 7, wherein the first medical device is a Huber needle with the first EMR element about a distal tip of a needle shaft of the Huber needle, the second medical device is a venous access port, the second EMR element, optionally, with one or more additional EMR elements, incorporated into the venous access port around a septum thereof, and the target location is a reservoir of the venous access port covered by the septum.

19. The medical-device navigation system of claim 18, wherein determining any movement of the first EMR through the external magnetic field with respect to the target location includes advancement of the distal tip of the Huber needle toward the septum of the venous access port by way of the relative location or velocity vectors between the first EMR element in the distal tip of the Huber needle, the second EMR element in the venous access port around the septum, and, optionally, the one or more additional EMR elements in the venous access port around the septum.

20. The medical-device navigation system of claim 19, the medical-device navigation processes further including displaying advancement of the distal tip of the Huber needle toward the septum of the venous access port with graphical representations of the distal tip of the Huber needle and the venous access port on a display screen of the console.
